(19) United States
(12) Patent Application Publication
(10) Pub. No.: US 2010/0113778 A1
(43) Pub. Date: May 6, 2010

(54) PROCESS FOR PREPARING O-CHLOROMETHYLPHENYLGLYOXYLIC ESTERS, IMPROVED PROCESS FOR PREPARING (E)-2-(2-CHLOROMETHYLPHENYL)-2-ALKOXIMINOACETIC ESTERS, AND NOVEL INTERMEDIATES FOR THEIR PREPARATION

(76) Inventors: John-Matthias Wiegand, Walding (AU); Karsten Luettgen, Plesching (AT); Wolfgang Skranc, Vienna (AT)

Correspondence Address: NIXON & VANDERHYE, PC, 901 NORTH GLEBE ROAD, 11TH FLOOR, ARLINGTON, VA, 22203, US

(21) Appl. No.: 12/595,113

(22) PCT Filed: Apr. 10, 2008
(86) PCT No.: PCT/EP2008/054354

§ 371 Date: Dec. 7, 2009

(30) Foreign Application Priority Data
Apr. 12, 2007 (AT) A 561/2007

(51) Int. Cl. C07D 265/30; C07C 229/34; C07C 69/635; C07C 237/20

(52) U.S. Cl. 544/162; 544/171; 560/39; 560/42; 560/51; 564/165

(57) ABSTRACT

An improved process for preparing o-chloromethylphenylglyoxylic esters of the formula

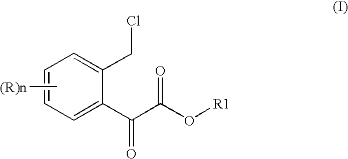

which comprises converting a compound of the formula

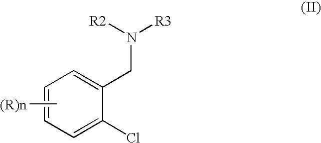

by reaction with magnesium into the corresponding Grignard reagent which is then reacted with a compound of the formula

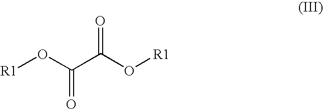

to give the compound of the formula

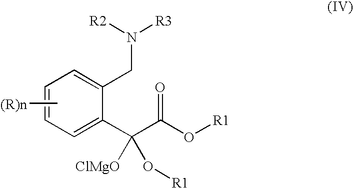

which is then cleaved by reaction with a chloroformic ester of the formula CICOOR4 or by reaction with phosgene to give the compound of the formula (I), followed by the isolation of the compound of the formula (I), and also an improved process for preparing (E)-2-(2-chloromethylphenyl)-2-alkoximinoacetic esters of the formula

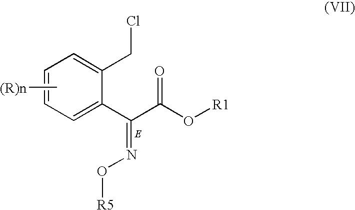

and intermediates for their preparation.

The present invention relates to an improved process for preparing o-chloromethylphenylglyoxylic esters, an improved process for preparing (E)-2-(2-chloromethylphenyl)-2-alkoximinoacetic esters and novel intermediates for preparing these esters.

o-Chloromethylphenylglyoxylic esters are important intermediates for preparing agrochemically active compounds or microbicides of the methoximinophenylglyoxylic ester series, as described, for example, in EP 0 254 426, EP 0 782 982, WO 95/18789 and WO 95/21153.

According to EP 0 782 982, for example, methyl o-(N,N-dimethylaminomethyl)phenylglyoxylate or methyl o-piperidinomethylphenylglyoxylate are obtained by reacting N-benzyldimethylamine and N-benzylpiperidine, respectively, with an organolithium compound, followed by reaction with a dialkyl oxalate compound, with a chloroformic ester to give the corresponding o-chloromethylphenylglyoxylic esters. Disadvantages of this reaction sequence are the use of expensive organolithium compounds and low temperatures of up to −50° C. required for this reaction, which make an industrial application difficult.

JP 2003-026640 discloses that 2-(morpholinomethyl)chlorobenzene, obtained by reaction of 2-chlorobenzyl chloride with morpholine, can be converted via a Grignard reaction and reaction with a dialkyl oxalate and to the corresponding o-(morpholinomethyl)phenylglyoxylic ester, which is then isolated from the reaction mixture by aqueous acidic work-up. This reaction sequence has the disadvantage of the aqueous acidic work-up of the basic amine product, which results in high product losses and/or large volumes to recover the product. (E)-2-(2-Chloromethylphenyl)-2-alkoximinoacetic esters are likewise important intermediates for preparing agrochemically active compounds or microbicides of the methoximinophenylglyoxylic ester series, as described, for example, in EP 0 254 426, EP 0 782 982, WO 95/18789 and WO 95/21153.

According to EP 0 782 982, for example, methyl (E)-2-(2-chloromethylphenyl)-2-methoximinoacetate is prepared by treating a solution of the (E/Z) isomer mixture of methyl 2-(2-chloromethylphenyl)-2-methoximinoacetate in methylcyclohexane with hydrogen chloride gas. This process has the disadvantage that a corrosive gaseous substance is used, which requires increased expense for apparatus.

It was an object of the present invention to provide an improved process for preparing o-chloromethylphenylglyoxylic esters which does not require low-temperature reaction conditions, does not require complicated and/or yield-reducing work-up steps and which affords the desired end products in high yield. It was another object of the present invention to provide an improved process for preparing (E)-2-(2-chloromethylphenyl)-2-alkoximinoacetic esters which does not require any corrosive gaseous substance.

Accordingly, the present invention provides an improved process for preparing o-chloromethylphenylglyoxylic esters of the formula

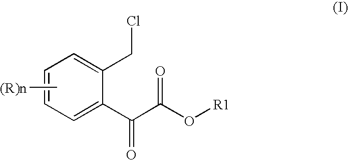

in which

R is a reaction-inert radical,

n is from 0 to 4 and

R1 may be a C₁-C₈-alkyl radical,

which comprises converting a compound of the formula

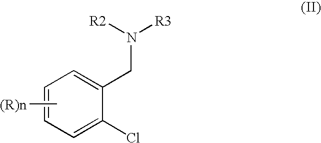

in which

n and R are as defined above, R2 and R3 independently of one another may be C₁-C₁₂-alkyl, C₁-C₁₂-alkenyl, C₁-C₁₂-alkoxyalkyl or C₃-C₆-cycloalkyl or R2 and R3 together with the nitrogen atom may be a 6- or 7-membered ring which, in addition to the nitrogen atom, may contain a further nitrogen atom or oxygen atom, by reaction with magnesium into the corresponding Grignard reagent which is then reacted with a compound of the formula

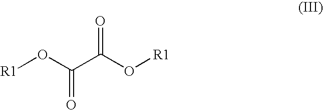

in which R1 is as defined above to give the compound of the formula

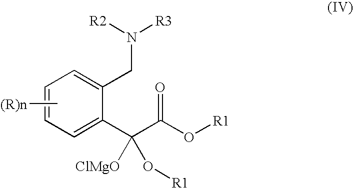

in which n, R, R1, R2 and R3 are as defined above, which is then cleaved by reaction with a chloroformic ester of the formula CICOOR4 in which R4 may be a C₁-C₈-alkyl radical or by reaction with phosgene to give the compound of the formula (I), followed by the isolation of the compound of the formula (I).

The process according to the invention is suitable for preparing o-chloromethylphenylglyoxylic esters of the formula (I).

In formula (I), R is a reaction-inert radical, i.e. the radical R can be chosen as desired, provided it is inert to the reaction conditions. Examples of such radicals are C₁-C₁₂-alkyl radicals, preferably C₁-C₆-alkyl radicals, C₁-C₁₂-alkenyl radicals, preferably C₁-C₆-alkenyl radicals, C₁-C₁₂-alkoxy radicals, preferably C₁-C₆-alkoxy radicals, phenyl, benzyl, nitro, etc.

n may be 0, 1, 2, 3 or 4; preferably, n=0.

R1 is a C₁-C₈-alkyl radical, such as, for example, methyl, ethyl, n-propyl, isopropyl, n-butyl, sec-butyl, tert-butyl, etc. Preferably, R1 is a C₁-C₂-alkyl radical, particularly preferably methyl.

The starting material used for the process according to the invention is a compound of the formula (II).

In formula (II), R and n are as defined above.

R2 and R3 independently of one another are C₁-C₁₂-alkyl, C₁-C₁₂-alkenyl, C₁-C₁₂-alkoxyalkyl or C₃-C₆-cycloalkyl.

Furthermore, R2 and R3 together with the nitrogen atom may be a 6- or 7-membered ring which, in addition to the nitrogen atom, may contain a further nitrogen atom or oxygen atom, preferably a 6-membered ring, particularly preferably morpholine.

Optionally substituted 2-(morpholinomethyl)chlorobenzenes of the formula (II) are known, for example, from JP 2003-026640 and can be prepared analogously to the process described in this publication from 2-chlorobenzyl chloride and morpholine.

In the first step of the process according to the invention, the compound of the formula (II) is reacted with magnesium to give the corresponding Grignard reagent.

To this end, 1 to 3 equivalents, preferably 1 to 1.5 equivalents, based on the compound of the formula (II), of magnesium are heated in a suitable solvent at a temperature of from 30° C. to 70° C., preferably from 40° C. to 60° C., and a catalytic amount of dibromoethane or iodine, about 0.01 to 0.5 equivalents, preferably 0.03 to 0.12 equivalents, is added.

Suitable solvents are, for example, ethers, such as, for example, diethyl ether, dibutyl ether, tert-butyl methyl ether, tetrahydrofuran, 1,4-dioxane, diethylene glycol dimethyl ether, etc., aromatic hydrocarbons, such as, for example, toluene, benzene, ethylbenzene, xylenes, etc., amines, such as, for example, triethylamine, pyridine, piperidine, etc., and mixtures of these. Particularly suitable is a mixture of THF and toluene.

The compound of the formula (II) is then added to the mixture obtained in this manner, and stirring is continued at from 60° C. to 130° C., preferably from 70° C. to 100° C., for a further 1 to 24 hours.

Magnesium which has not been consumed is then filtered off, and—if a solvent mixture has been used which, as polar component, contains an ether, such as, for example, THF, diethyl ether, 1,4-dioxane, etc., which gives unwanted by-products in the subsequent reaction with the compound of the formula (III)—a solvent exchange for an unpolar solvent, such as, for example, dibutyl ether, tert-butyl methyl ether, diethylene glycol dimethyl ether, toluene, benzene, ethylbenzene, xylenes, etc., is carried out.

The solution of the Grignard reagent obtained in this manner is, in the second step of the process according to the invention, at a reaction temperature of from −20° C. to +20° C., preferably from −10° C. to +10° C., added to a solution, cooled to from −20° C. to +10° C., preferably to from −10° C. to +5° C., of the compound of the formula (III).

Here, the compound of the formula (III) is employed in an amount of from 1 to 3 equivalents, preferably from 1.1 to 2 equivalents and particularly preferably from 1.3 to 1.7 equivalents, based on the compound of the formula (II).

Suitable solvents are, for example, dibutyl ether, tert-butyl methyl ether, diethylene glycol dimethyl ether, toluene, benzene, ethylbenzene, xylenes, etc., and also mixtures of these; toluene is particularly suitable.

After the addition has ended, the reaction temperature is kept at this temperature for some time (from 10 minutes to 10 hours, preferably from 30 minutes to 5 hours, particularly preferably from 1 to 2 hours) and then slowly (over a period of several hours) brought to room temperature.

The suspension obtained in this manner, which comprises the compound of the formula (IV), is then used directly and without further work-up for the next step.

Aminoketo ester Mg acetals of the formula (IV) and their use for preparing agrochemically active compounds are novel, and thus also part of the subject matter of the present invention.

By aqueous work-up of the resulting suspension, which comprises the compound of the formula (IV), it is possible to isolate the corresponding o-aminomethylphenylglyoxylic esters. In particular, methyl o-morpholinemethylphenylglyoxylate of the formula

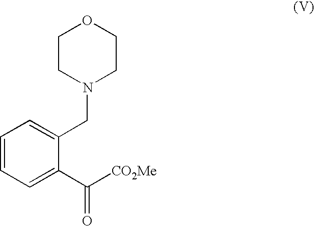

and its use for preparing agrochemically active compounds are novel, and thus also form part of the subject matter of the present invention.

The compound of the formula (V) can be converted by oximation, as described, for example, in EP 0 254 426 and EP 0 782 982, into the corresponding methyl 2-(2-morpholinomethylphenyl)-2-methoximinoacetate of the formula

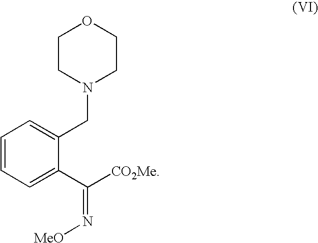

The compound of the formula (VI) and its use for preparing agrochemically active compounds are novel, and thus also part of the subject matter of the present invention.

In the third step of the process according to the invention, a chloroformic ester of the formula CICOOR4 or phosgene is added to the suspension which comprises the compound of the formula (IV). Here, R4 is a C₁-C₈-alkyl radical. Preferably, R4 is methyl or ethyl, particularly preferably methyl.

The chloroformic ester of phosgene is employed in an amount of from 1 to 5 equivalents, preferably from 1.5 to 3 equivalents, based on (II).

The reaction mixture is then heated at 50° C. to 120° C., preferably from 70° C. to 110° C., for a period of from 30 minutes to 6 hours, preferably from 1 to 3 hours.

After cooling of the reaction mixture to room temperature, an aqueous acidic work-up is carried out for removing the cleavage product from the desired compound of the formula (I).

Using the process according to the invention, the desired o-chloromethylphenylglyoxylic esters of the formula (I) are obtained in a simple manner in high yields and purities.

The o-chloromethylphenylglyoxylic esters of the formula (I) prepared according to the invention are highly suitable for preparing the corresponding 2-(2-chloromethylphenyl)-2-alkoximinoacetic esters by oximation, as described, for example, in EP 0 254 426 and EP 0 782 982. These compounds are obtained as an E/Z mixture.

Accordingly, the present invention also provides an improved process for preparing (E)-2-(2-chloromethylphenyl)-2-alkoximinoacetic esters of the formula

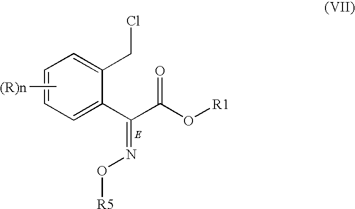

in which

R is a reaction-inert radical,

n is from 0 to 4 and

R1 and R5 independently of one another may be C₁-C₈-alkyl radicals,

which comprises treating a compound of the formula

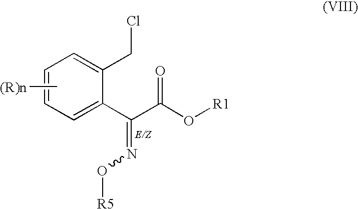

in which n, R, R1 and R5 are as defined above with an aqueous mineral acid.

The process according to the invention is suitable for preparing (E)-2-(2-chloromethylphenyl)-2-alkoximinoacetic esters of the formula (VII).

In formula (VII), R is a reaction-inert radical, i.e. the radical R can be chosen as desired, provided it is inert to the reaction conditions. Examples of such radicals are C₁-C₁₂-alkyl radicals, preferably C₁-C₆-alkyl radicals, C₁-C₁₂-alkenyl radicals, preferably C₁-C₆-alkenyl radicals, C₁-C₁₂-alkoxy radicals, preferably C₁-C₆-alkoxy radicals, phenyl, benzyl, nitro, etc.

n may be 0, 1, 2, 3 or 4; preferably, n=0.

R1 and R5 independently of one another are C₁-C₈-alkyl radicals, such as, for example, methyl, ethyl, n-propyl, isopropyl, n-butyl, sec-butyl, tert-butyl, etc. Preferably, R1 and R5 independently of one another are C₁-C₂-alkyl radicals, particularly preferably methyl.

The starting material used for the process according to the invention is a compound of the formula (VIII) in which n, R, R1 and R5 are as defined above.

Compounds of the formula (VIII) are known and can be prepared by oximation of o-chloromethylphenylglyoxylic esters of the formula (I), as described, for example, in EP 0 254 426 and EP 0 782 982.

In the process according to the invention, the compound of the formula (VIII) is reacted with an aqueous mineral acid to give the compound of the formula (VII).

To this end, from 0.5 to 20 equivalents, preferably from 1 to 10 equivalents, based on the compound of the formula (VIII), of mineral acid are heated in the presence of a suitable solvent at a temperature of from 20° C. to 100° C., preferably from 60° C. to 90° C.

Suitable mineral acids are, for example, hydrochloric acid, sulfuric acid, phosphoric acid, nitric acid, etc. Hydrochloric acid is particularly suitable. Suitable solvents are, for example, hydrocarbons, such as, for example, pentane, hexane, heptane, etc., and also aromatic hydrocarbons, such as, for example, toluene, benzene, ethylbenzene, xylenes, etc., and also mixtures of these. Toluene is particularly suitable.

The organic phase is then separated off, the solvent is removed and the residue, which contains the compound of the formula (VII), is crystallized from a suitable solvent.

Suitable solvents are, for example, alcohols, such as methanol, ethanol, propanol, isopropanol, etc., hydrocarbons, such as, for example, pentane, hexane, heptane, etc., and also aromatic hydrocarbons, such as, for example, toluene, benzene, ethylbenzene, xylenes, etc., and also mixtures of these. Methanol is particularly suitable.

A particularly useful compound of Formula VII is CLMO, wherein n=0 and wherein R1 and R5 are methyl.

Strobilurines are a type of fungicides that inhibit the respiratory system of the fungi. Strobilurines may be synthesized starting from compounds according to Formula VII. The known strobilurines Kresoxim-methyl and Dimoxystrobin, respectively may be synthesized from CLMO by substitution reaction with the respective phenol as shown in examples 7 and 8. Variations on this synthesis may easily be employed by the skilled person using the disclosure of the present invention.

### EXAMPLE 1

### Synthesis of 2-Chlorobenzylmorpholine (IIa) (not Subject Matter of the Invention)

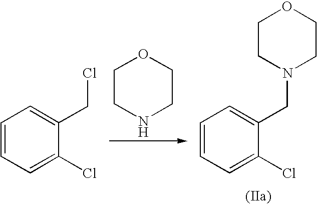

2-Chlorobenzyl chloride (1 eq., 200 g) was dissolved in toluene (400 ml), 15% strength NaOH (1.3 eq., 430 g) and morpholine (1.2 eq., 130 g) were added and the mixture was heated at the boil for 16 hours. After cooling to room temperature, the phases were separated, the organic phase was washed with 200 ml of water and the phases were again separated. Subsequently, the organic phase was dried by azeotropic distillation and the solvent was removed completely under reduced pressure. This gave 2-(morpholinomethyl)chlorobenzene (IIa) (260 g, yield: 98.9%) in the form of a colorless oil.

### EXAMPLE 2

### Synthesis of Methyl o-morpholinomethylphenylglyoxylate Methylmagnesium Chloride Acetal (IVa) by Grignard Reaction of the Compound of the Formula (IIa) and Reaction with Dimethyl Oxalate (Subject Matter of the Invention)

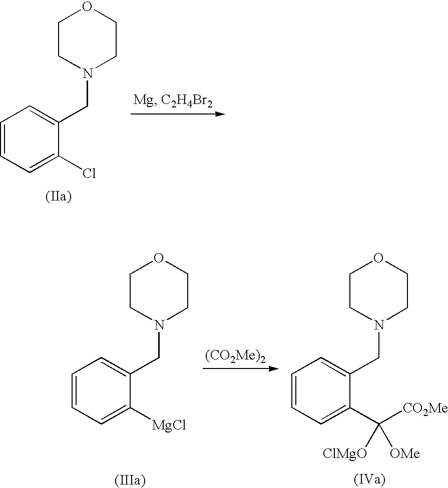

Magnesium (1.1 eq., 10.56 g) was added to a mixture of toluene (130 ml) and THF (100 ml), and the mixture was heated to 50° C. with stirring. Dibromoethane (0.1 eq., 7.52 g) was then added slowly, and after a few minutes a strong exothermic reaction with vigorous foaming could be observed. After the exothermic reaction had subsided, the mixture was heated to the boil (about 83° C.), and the temperature was maintained for 5 minutes. At the boil, 2-(morpholinomethyl)chlorobenzene (IIa) (1.0 eq., 84.56 g) was added dropwise over a period of 5 minutes, and the mixture was kept at the boil. 15 minutes after the addition of 2-(morpholinomethyl)chlorobenzene (IIa) had ended (temperature of the reaction mixture about 90° C.), THF (30 ml) was added, and the temperature of the mixture decreased to about 86° C. One hour after the end of the addition of 2-(morpholinomethyl)chlorobenzene, THF (50 ml) was added, the temperature of the reaction vessel was adjusted to 65° C. and the mixture was kept at this temperature for 12 hours. The resulting brown solution of the compound of the formula (IVa) was filtered through a G3 frit and, at 50° C. and under reduced pressure, concentrated to 300 ml. In the solvent exchange that followed, twice toluene (100 ml) was added and in each case the same amount of solvent mixture was distilled off at 50° C. under reduced pressure. This gave a brown viscous oil which was cooled to 0° C. and then, over a period of one hour, added dropwise to a solution, cooled to −5° C., of dimethyl oxalate (1.5 eq., 70.80 g) in toluene (375 ml). After the addition had ended, the temperature was kept at −5° C. for two hours and then, over a period of 12 hours, increased to room temperature. The resulting yellowish suspension of the compound of the formula (IVa) was used without further treatment in the next step (Example 3). Characterization: IR: v=3295, 1641, 1321 cm⁻¹.

### EXAMPLE 3

### Methyl o-morpholinemethylphenylglyoxylate (V) (Subject Matter of the Invention)

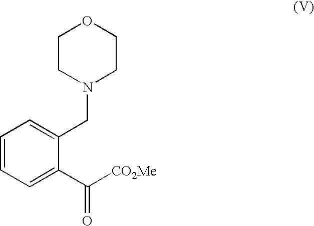

The compound of the formula (V) can be isolated by aqueous work-up of the suspension of the compound of the formula (IVa). Characterization: ¹H-NMR (300 MHz, CDCl₃): δ=2.38 (t, 4H), 3.59 (t, 6H), 3.88 (s, 3H), 7.17-7.44 (m, 4H).

### EXAMPLE 4

### Methyl 2-(2-morpholinomethylphenyl)-2-methoximinoacetate (VI) (Subject Matter of the Invention)

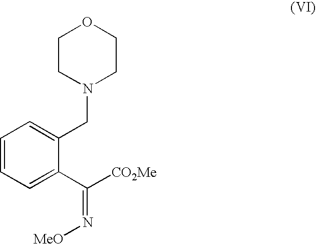

By oximation of the compound of the formula (V), for example as described in EP 0 254 426 and EP 0 782 982, it is possible to isolate the compound of the formula (VI). Characterization: ¹H-NMR (400 MHz, CDCl₃): δ=2.32-2.34 (d, 4H), 3.56-3.59 (m, 4H), 3.88 (s, 2H), 3.90 (s, 3H), 3.99 (s, 3H), 7.12-7.33 (m, 4H).

### EXAMPLE 5

### Synthesis of Methyl o-chloromethylphenylglyoxylate (Ia) by Reaction of the Compound of the Formula (IVa) with Methyl Chloroformate (Subject Matter of the Invention)

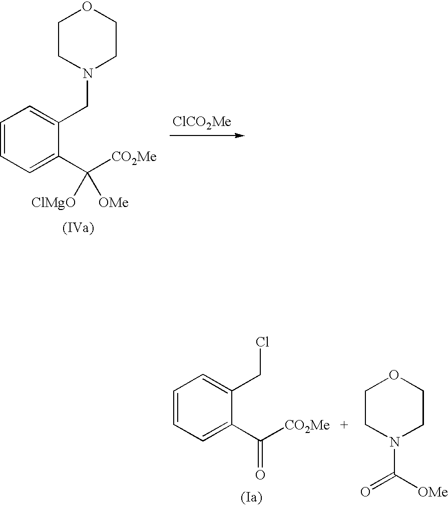

Methyl chloroformate (2.0 eq., based on the compound of the formula (IIa), 76.8 g) was added to the suspension, obtained in example 2, of the compound of the formula (IVa) in toluene, and the mixture was heated in a closed reaction vessel (autoclave) at 100° C. for two hours, and a pressure increase to 12 bar was observed. After the reaction mixture had cooled to room temperature, the resulting brown suspension was washed twice with in each case 100 ml of conc. HCl and once with 100 ml of water, the organic phase was dried over Na₂SO₄ and the solvent was removed under reduced pressure. This gives methyl o-chloromethylphenylglyoxylate (Ia) (53.7 g, content: about 48% (NMR), yield: 30.3% (starting from the compound of the formula (IIa)) in the form of a brown oil.

### EXAMPLE 6

### Synthesis of Methyl (E)-2-(2-chloromethylphenyl)-2-methoximinoacetate (VIIa) by Reaction of the Compound of the Formula (Ia) with Methoxyamine Hydrochloride (not Subject Matter of the Invention) and (E/Z)-Isomerization with Aqueous HCl (Subject Matter of the Invention)

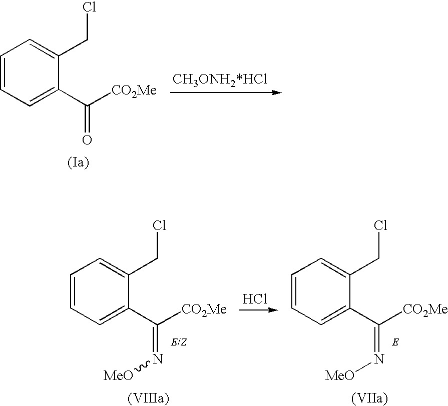

The brown oil of the compound of the formula (Ia) obtained in example 3 was dissolved in methanol (100 ml), a 30% strength solution of methoxyamine hydrochloride in H₂O (2.0 eq., based on the compound of the formula (Ia), 70.0 g), which had been adjusted to pH=3 beforehand, was added, and the mixture was stirred at 55° C. for 1.5 hours. The reaction mixture was cooled and extracted twice with toluene (100 ml). The combined organic phases, which contain the compound of the formula (VIIIa), were washed twice with conc. hydrochloric acid (20 ml), conc. hydrochloric acid (50 ml) was then added and the mixture was stirred at 80° C. for 2 hours. After the reaction mixture had cooled to room temperature, the phases were separated, the organic phase was washed once with water (50 ml), the phases were separated and the organic phase was freed from the solvent under reduced pressure. The oily residue was taken up in methanol (30 ml) and cooled at −18° C. for 16 hours, and the resulting precipitated crystals of methyl 2-(2-chloromethylphenyl)-2-methoximinoacetate (VIIa) were isolated (11.63 g, yield: 73%, E/Z isomer ratio:about 99:1).

### EXAMPLE 7

### Synthesis of Kresoxim-Methyl from CLMO

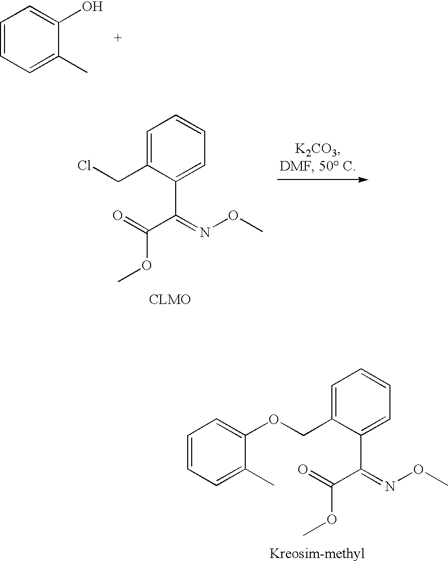

To a mixture of o-Kresol (2.0 g, 18 mmol) and potassium carbonate (6.0 g, 43 mmol) in DMF (6 mL), a solution of CLMO in DMF (45 w/w %, 9.7 g, 18 mmol) was added at room temperature. Then the suspension was heated to 50° C. upon stirring for 64 hours. Water (100 mL) was subsequently added and the aqueous phase was extracted with ethyl acetate (3×70 mL). The combined organic phases were washed with water (1×100 mL), dried over sodium sulfate and then volatile compounds were evaporated in vacuo to give an orange-red oil (5.4 g, 59 area % Kresoxim-methyl according to GC). The product was purified by medium-pressure column chromatography using silica 60 and heptane/ethyl acetate (7/1) as eluent. Product-containing fractions were combined and dried in vacuo to give Kresoxim-methyl as a white solid (0.5 g).

GC: 99 area % Kresoxim-methyl.

GC/MS (EI): [M]+ found (313).

¹H NMR (CDCl₃) δ=7.58 (d, 1H, J=7.6 Hz, Hₐᵣₒₘ), 7.47-7.37 (m, 2H, Hₐᵣₒₘ), 7.21 (dd, 1H, J₁=7.6 Hz, J₂=1.2 Hz, Hₐᵣₒₘ), 7.12 (m, 2H, Hₐᵣₒₘ), 6.85 (m, 1H, Hₐᵣₒₘ), 6.77 (d, 1H, J=8.4 Hz), 4.95 (s, 2H, CH₂), 4.01 (s, 3H, NO—CH₃), 3.82 (s, 3H, COOCH₃), 2.25 (s, 3H, Ar—CH₃).

¹³C NMR (CDCl₃) δ=163.3 (C═O), 156.6, 149.4 (C═N), 135.8, 130.8, 130.7, 129.7, 129.0, 128.6, 127.6, 127.0, 126.8, 120.7, 111.2 (all Cₐᵣₒₘ), 68.1 (CH₂), 63.9 (NOCH₃), 53.0 (COOCH₃), 16.31 (Ar—CH₃).

### EXAMPLE 8

### Synthesis of Dimoxystrobin from CLMO

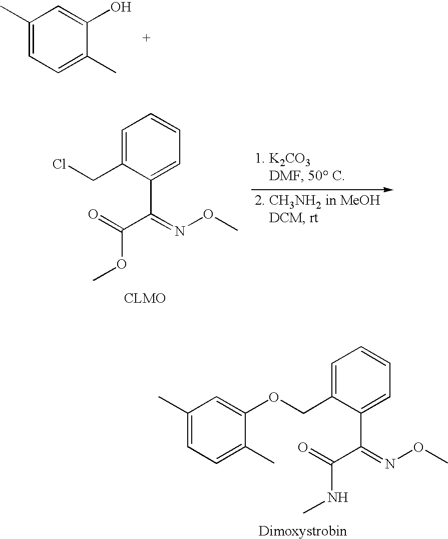

To a mixture of 2,5-dimethylphenol (2.0 g, 16 mmol) and potassium carbonate (5.3 g, 38 mmol) in DMF (6 mL), a solution of CLMO in DMF (45 w/w %, 9.7 g, 18 mmol) was added at room temperature. Then the suspension was heated to 50° C. upon stirring for 64 hours. Water (100 mL) was subsequently added and the aqueous phase was extracted with ethyl acetate (3×70 mL). The combined organic phases were washed with water (1×100 mL), dried over sodium sulfate and then volatile compounds were evaporated in vacuo to give an orange-red solid (5.3 g, 63 area % “Kresoxim-dimethyl” according to GC). The product was purified by medium-pressure column chromatography using silica 60 and heptane/ethyl acetate (7/1) eluent. Product-containing fractions were combined and dried in vacuo to give “Kresoxim-dimethyl” as a white solid (1.2 g).

GC: 97 area % Kresoxim-methyl.

GC/MS (EI): [M]⁺ found (327).

¹H NMR (CDCl₃) δ=7.59 (d, 1H, J=7.6 Hz, Hₐᵣₒₘ), 7.46-7.35 (m, 2H, Hₐᵣₒₘ), 7.20 (dd, 1H, J₁=7.4 Hz, J₂=1.2 Hz, Hₐᵣₒₘ), 7.00 (d, 1H, J=7.6 Hz, Hₐᵣₒₘ), 6.66 (d, 1H, J=7.2 Hz), 6.58 (s, 1H, Hₐᵣₒₘ), 4.92 (s, 2H, CH₂), 4.03 (s, 3H, NO—CH₃), 3.82 (s, 3H, COOCH₃), 2.28 (s, 3H, Ar—CH₃), 2.25 (s, 3H, Ar—CH₃).

¹³C NMR (CDCl₃) δ=163.2 (C═O), 156.4, 149.3 (C═N), 136.4, 135.8, 130.7, 130.4, 129.6, 128.9, 128.6, 128.4, 127.5, 123.7, 121.1, 112.1 (all Cₐᵣₒₘ), 67.9 (CH₂), 63.8 (NOCH₃), 52.9 (COOCH₃), 21.4 (Ar—CH₃), 15.8 (Ar—CH₃).

“Kresoxim-dimethyl” (1.0 g, 3.1 mmol) was dissolved in dichloromethane (5 mL) and then methyl amine in methanol (24 w/w %, 2 mL) was added to the solution. The resulting mixture was stirred at room temperature. After 3 hours again methyl amine in methanol (24 w/w %, 2 mL) was added and the solution was allowed to stir at room temperature over the weekend. All volatile compounds were removed in vacuo to give the product Dimoxystrobin as a yellowish solid (0.73 g, 73%).

GC: 98 area % Kresoxim-methyl.

GC/MS (EI): [M]⁺ found (326).

1.-10. (canceled)

11. An improved process for preparing o-chloromethylphenylglyoxylic esters of the formula 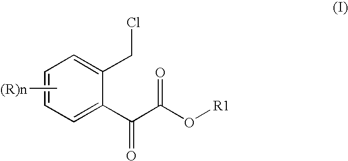
in which
R is a reaction-inert radical,
n is from 0 to 4 and
R1 may be a C₁-C₈-alkyl radical,
which comprises converting a compound of the formula 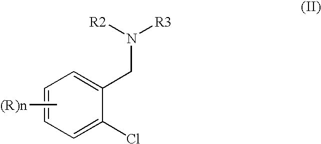
in which
n and R are as defined above,
R2 and R3 independently of one another may be C₁-C₁₂-alkyl, C₁-C₁₂-alkenyl, C₁-C₁₂-alkoxyalkyl or C₃-C₆-cycloalkyl or R2 and R3 together with the nitrogen atom may be a 6- or 7-membered ring which, in addition to the nitrogen atom, may contain a further nitrogen atom or oxygen atom, by reaction with magnesium into the corresponding Grignard reagent which is then reacted with a compound of the formula 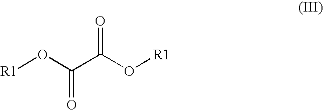
in which R1 is as defined above to give the compound of the formula 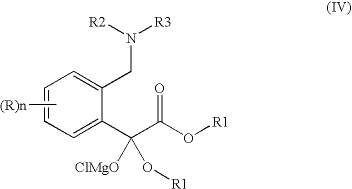
in which n, R, R1, R2 and R3 are as defined above, which is then cleaved by reaction with a chloroformic ester of the formula CICOOR4 in which R4 may be a C₁-C₈-alkyl radical or by reaction with phosgene to give the compound of the formula (I), followed by the isolation of the compound of the formula (I).

12. The process as claimed in claim 11 wherein in the first step a solvent from the group of the ethers, the aromatic hydrocarbons or the amines or mixtures thereof is used.

13. The process as claimed in claim 11 wherein the compound of the formula (IV) is used directly as a suspension for the reaction with the chloroformic ester or phosgene.

14. A compound of the formula. 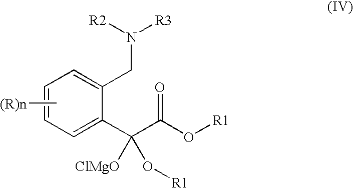
in which n, R, R1, R2 and R3 are as defined in claim 11.

15. The compound of the formula 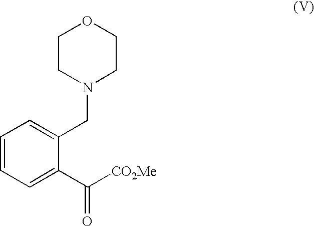
obtained by aqueous work-up of the compound of the formula (IV) in which n=0, R1 is methyl and R2 and R3 together with the nitrogen atom form a six-membered ring which additionally contains an oxygen atom.

16. The compound of the formula 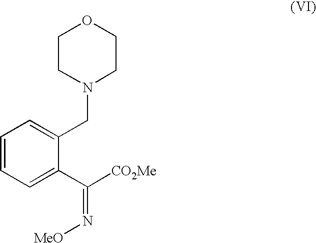
obtained by oximation of the compound of the formula (V), and its use for preparing agro chemically active compounds.

17. An improved process for preparing (E)-2-(2-chloromethylphenyl)-2-alkoximinoacetic esters of the formula 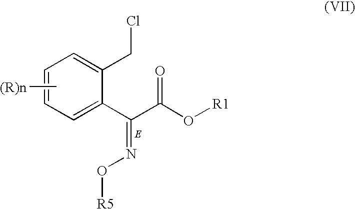
in which
R is a reaction-inert radical,
n is from 0 to 4 and
R1 and R5 independently of one another may be C₁-C₈-alkyl radicals,
which comprises treating a compound of the formula 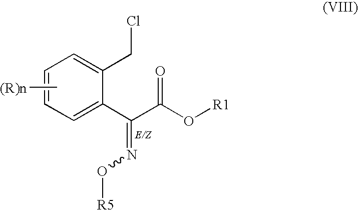
in which n, R, R1, and R5 are as defined above, obtained by oximation of the compound of the formula (I), with an aqueous mineral acid.

18. The process as claimed in claim 17, wherein the mineral acid used is hydrochloric acid, sulfuric acid, phosphoric acid or nitric acid.

19. Use of the compounds of formulas IV, V and VI for preparing agro chemically active compounds.

20. Process for preparing compounds of formula IX 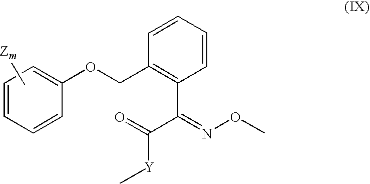
in which
Y is NH or O
Z is a C₁-C₄ alkyl group
m is 0 to 5
by reacting a compound of formula I produced according to claim 11 with a compound of formula X 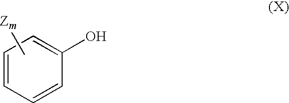
in which Z and m are defined as above in the presence of an alkalicarbonate and optionally oximating the resulting compound.
